(19) United States
(12) Patent Application Publication
(10) Pub. No.: US 2022/0402767 A1
(43) Pub. Date: Dec. 22, 2022

(54) METHOD FOR THERMAL BARRIER COATING PRODUCTION BY RECYCLING THERMAL BARRIER COATING MATERIALS

(71) Applicant: Xylon Technical Ceramics, Inc., Alfred, NY (US)

(72) Inventors: Nicholas H. Burlingame, Belmont, NY (US); Samuel J. Burlingame, Belmont, NY (US)

(73) Assignee: Xylon Technical Ceramics, Inc., Alfred, NY (US)

(21) Appl. No.: 17/838,584

(22) Filed: Jun. 13, 2022

Related U.S. Application Data
(60) Provisional application No. 63/211,104, filed on Jun. 16, 2021.

(51) Int. Cl. C01F 17/10; C01G 25/00; C01G 25/02; C01G 25/06; C07C 51/41; C22B 59/00; C22B 34/14

(52) U.S. Cl. CPC C01F 17/10; C01G 25/00; C01G 25/02; C01G 25/06; C07C 51/41; C22B 59/00; C22B 34/14

(57) ABSTRACT

Disclosed are methods to produce Thermal Barrier Coating (TBC) products using materials recycled from TBC waste. These methods include ways to produce zirconium and rare earth chemicals and raw materials appropriate for producing TBC materials.

This application claims priority under 35 U.S.C. § 119(e) from U.S. Provisional Patent Application No. 63/211,104, for a “METHOD FOR THERMAL BARRIER COATING PRODUCTION BY RECYCLING THERMAL BARRIER COATING MATERIALS,” filed Jun. 16, 2021 by Nicholas H. Burlingame and Samuel J. Burlingame, which is hereby incorporated by reference in its entirety.

Disclosed herein are methods to produce thermal barrier coating (TBC) materials by recovering and recycling rare earth and zirconium constituents from TBC wastes.

### CROSS-REFERENCE

Cross-reference is made to the following related patents, which are also hereby incorporated by reference in their entirety:
U.S. Pat. No. 8,940,256 B2, granted Jan. 27, 2015, for a Method for Recycling of Rare Earth and Zirconium Oxide Materials, by Nicholas H. Burlingame and Samuel Burlingame (discloses the use of ammonium sulfate to digest rare earth oxide and zirconium oxide to form a soluble salt);
U.S. Pat. No. 5,418,003 A, granted May 23, 1995, for Vapor Deposition of Ceramic Materials, by Robert W. Bruce et al. (discloses the chemical purity and structural guidelines for producing useful EB-PVD ingot); and
U.S. Pat. No. 6,117,560 A, granted Sep. 12, 2000, for Thermal Barrier Coating Systems and Materials, by Michael J. Maloney (discloses the use of family of pyrochlore materials for thermal barrier coatings).

### BACKGROUND AND SUMMARY

Coatings for aviation applications represent several of the most carefully developed advanced materials groups. Thermal barrier coatings (TBCs) are integral for both commercial and military aircraft, as these coatings directly impact engine fuel efficiency, use temperatures, and operational conditions. The materials utilized for these coatings contain precise formulations, with the most prevalent TBCs being comprised mainly of rare earth oxides and zirconia. Furthermore TBCs are applied through highly technical coating processes such as plasma spray and Electron Beam Physical Vapor Deposition (EB-PVD) processes. These TBC application methods are typically exceedingly inefficient, with deposition rates of as low as 10-20%. The low deposition efficiencies of the TBC coating process results in large quantities of the input TBC materials becoming waste. While it would be sensible to reuse the waste materials in the production of new TBC products, there are numerous factors which may prevent the direct reuse of TBC wastes. Material properties are altered during the EB-PVD and plasma spray application processes, resulting in waste materials which may be functionally different from the input TBC materials; additionally, methods for collecting the TBC wastes cannot be accomplished without imparting significant amounts of impurities to the wastes. Consequently, any efforts to use TBC wastes to produce new TBC materials would require implementation of a recycling process capable of removing any unwanted contaminants and creating recycled materials with the necessary properties to facilitate reuse in TBC applications.

In the embodiments disclosed herein recycled TBC waste is employed in the production of raw materials for TBC EB-PVD materials, EB-PVD ingots, TBC plasma spray materials and plasma spray powders. The basics of the method include one or more of the following: (a) recycling TBC waste materials to form recycled rare earth and zirconium products of sufficiently high purity and quality to be used to produce TBC products; (b) using recycled rare earth and zirconium products recovered from TBC wastes to produce EB-PVD ingots; and (c) using recycled rare earth and zirconium products recovered from TBC wastes to produce TBC plasma spray powders.

Disclosed in embodiments herein is a method for recovery of thermal barrier coating materials from waste comprising: collecting and classifying TBC waste materials to determine chemical composition and impurity levels; processing the TBC waste to convert it to a fine powder; reacting the fine powder to form a soluble zirconium- and rare earth-containing material; and collecting the soluble zirconium and rare earth containing materials, including recycled zirconium and rare earth materials, which consist essentially of precursor materials suitable for use in producing TBC feedstock materials.

### BRIEF DESCRIPTION OF THE DRAWING

FIG. 1 is an illustrative representation of a method for producing thermal barrier coating materials by recovering and recycling rare earth and zirconium constituents from thermal barrier coating wastes, and operations associated therewith.

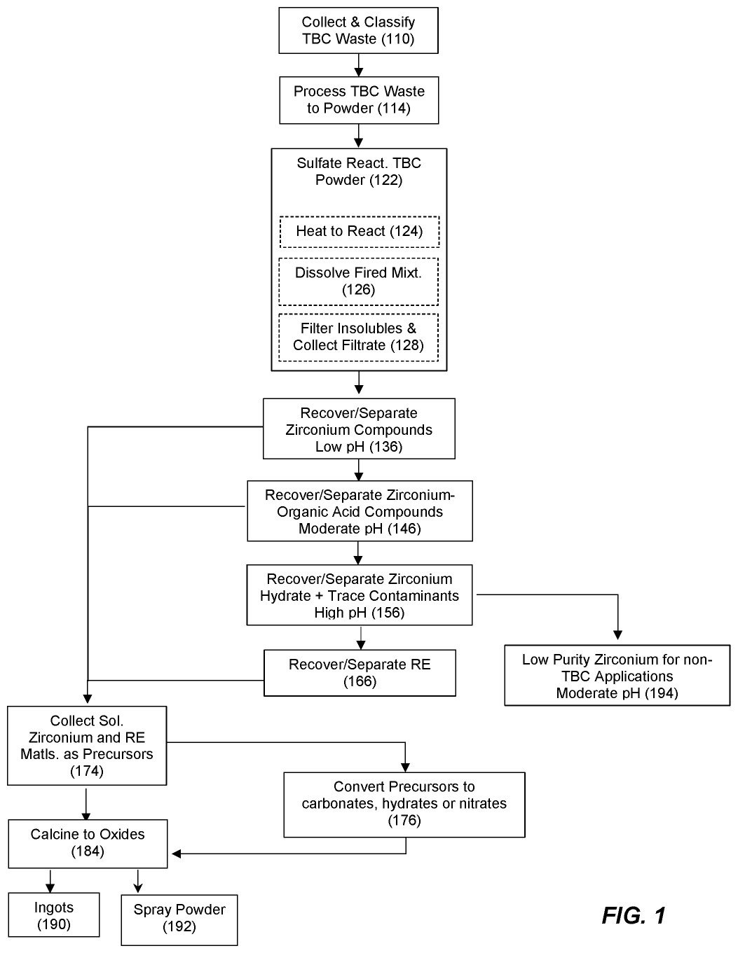

The various embodiments described herein are not intended to limit the disclosure to those embodiments described. On the contrary, the intent is to cover all alternatives, modifications, and equivalents as may be included within the spirit and scope of the various embodiments and equivalents set forth. For a general understanding, reference is made to the drawings. In the drawings, like references have been used throughout to designate identical or similar elements. It is also noted that the drawings may not have been drawn to scale and that certain regions may have been purposely drawn disproportionately so that the features and aspects could be properly depicted.

### DETAILED DESCRIPTION

The following acronyms and keywords are used in the description:
EB-PVD Coating: Coating applied by EB-PVD process where input feed material is evaporated via electron beam and material condensate is deposited on a coating target;
EB-PVD Ingot: Input feed material for EB-PVD coating process;
Plasma Spray Coating: Coating applied by spraying and melting a material passed through a thermal plasma;
Rare Earth Elements: Elements comprising the fifteen lanthanides plus Scandium and Yttrium;
REO: Rare Earth Oxide;
RE: Rare Earth; and
Zirconia: Zirconium Oxide

The FIGURE is a flow diagram representing an exemplary processing method as disclosed herein. The process steps encompassed by the method may include the following. Collecting and classifying TBC waste materials (110) to determine chemical composition and impurity levels. This operation is important so that subsequent processing can directly address impurities or waste materials that are outside of typical process parameters.

Next, physically and/or mechanically processing (114) the TBC waste to clean and convert it to a fine powder. Waste may be in the form of powder, large aggregates, or slurry. Processing of incoming waste may include one or more of grinding, milling (e.g., ball milling), crushing, etc. The use of physical or mechanical processing reduces the size of material pieces, perhaps reducing to particle size, to allow initial cleaning to be done to TBC waste materials to remove any easily separable portion of impurities. For example, magnetic removal of tramp iron or other ferromagnetic materials may be performed. Other preparation processing that is available includes acid leaching and water rinse to remove water soluble contaminants or ultra-fine powder contaminants.

Once the preparation of TBC waste material is performed, then preparation of reaction feedstock material is conducted. Generally the reaction feedstock is in the form of a powder that may be produced by progressively finer grinding/sieving or the like. Processing the TBC waste to convert it to a fine power includes physically or mechanically altering the waste material to produce a powder. For example, of 20 to 325 (U.S. Mesh), yielding particles in the range of 841 microns to 44 microns, and in particular a 100 mesh (149 microns or less) provided appropriate particles for further processing. Once the powder is formed, it is then reacted to form a soluble zirconium- and rare earth-containing material using thermal and/or chemical means, and in particular, a chemical means may include using the sulfate reaction process described in U.S. Pat. No. 8,940,256 B2, previously incorporated by reference.

Use of the sulfate reaction process comprises the steps of:
a) Dissolve the water soluble zirconium- and rare earth-containing material in de-ionized water to form a solution containing both zirconium and rare earth constituents (solution pH should be <1);
b) Remove any insoluble materials present with reaction solution via sedimentation, filtration or other means;
c) Adjust solution pH and chemistry to extract zirconium compounds at low pH (for example, a pH at or below 2-3);
d) Collect and process zirconium compounds to yield materials suitable for use in TBC materials;
e) Adjust solution pH and chemistry to extract residual contaminants and remove contaminants from solution;
f) Adjust solution pH and chemistry to extract rare earth compounds; and
g) Collect and process rare earth compounds to yield materials suitable for use in TBC materials.
   Through this method the collected zirconium and rare earth materials will have been separated from impurities and contaminants considered detrimental to TBC material products. And, the collected zirconium and rare earth materials will have been recovered at sufficiently high purities, and exhibit properties such that they are suitable for use in the further production of TBC materials. In general, the types or sources of water employed in the various steps may be tap water, de-ionized (DI) water, reverse-osmosis (RO) water, etc. However, it should be understood that mineral contaminants from tap water may impact or cause undesirable reactions, so while more costly a safe approach is to use DI water, at least in critical steps.

Recycled zirconium and rare earth materials will consist of appropriate precursors materials for use in producing TBC feedstock materials for EB-PVD ingots and/or plasma spray powders. The EB-PVD ingot feedstock materials can be pressed into ingots, fired, and machined to form finished EB-PVD ingots. And, plasma spray feedstock materials can be processed into appropriate plasma spray powder materials by thermal and/or chemical methods that include chemical precipitation, agglomeration and sintering, electric arc fusion, and induction fusion.

Having generally described a method for recovery of thermal barrier coating materials from waste, the following description is directed to a more detailed disclosure of recovering and recycling rare earth and zirconium constituents from TBC wastes to for usable TBC materials. First, it is important to consider the types of waste that may be suitable for recovery.

Examples of waste materials and sources that may be suitable for processing in accordance with the methods and operations disclosed herein include (a) waste collected from EB-PVD coating chamber; (b) waste collected from plasma spray coating chamber, (c) waste collected from dust collection systems associated with coating chambers, (d) waste in the form of powder, large aggregates, or slurry, (e) waste containing additional contaminants including powder or solid pieces of metal or metal oxides; and (f) waste containing mixtures of multiple TBC materials collected together. It should also be understood that the different waste sources may justify distinct pre-treatments. For example, waste that is known to include metal or metal fragments may justify additional processing, depending upon the nature of the metal (e.g., a magnetic separator may be employed for ferromagnetic particles).

Once the powdered form of reaction feedstock is available, the processing continues with the reaction step (122), where waste materials are mixed with other reactive materials, for example, ammonium sulfate. The ammonium sulfate and ground feedstock material can be further milled or pulverized to reduce particle size, an action that increases reactivity as the finer particles facilitate more comprehensive and intimate mixing between the sulfate and waste feedstock. In one embodiment, ammonium sulfate is mixed with waste material at a ratio of 1.0 to 6.0 moles of ammonium sulfate to 1.0 mole of TBC waste material, or more preferably 1.5 to 3.0 moles of ammonium sulfate to 1.0 mole of TBC waste material. Moreover, excess ammonium sulfate may be used when oxide waste is coarse and pulverizing is not practical, or when removing material from a substrate (e.g., removing TBC from a turbine blade or fragments thereof). The mixture of ammonium sulfate and feedstock waste can be processed as a powder mix, and may optionally be further processed through the use of pelletizing, extruding, or other methods of consolidation. Consolidation of the mixture promotes a more intimate mixing between the sulfate and waste constituents, resulting in the reaction efficiency being greatly enhanced. Other alternative methods for creating reaction feedstock may employ sulfuric roast and alkali roast.

Once the mixture of ammonium sulfate and feedstock waste is available, it is heated or fired under conditions sufficient to initiate a reaction (124) and decompose the waste into a product that is, to a large degree, soluble in an aqueous solution. The optimal firing range temperature is from about 350° C. to about 500° C.

Next, the fired/reacted mixture is dissolved in water to yield an aqueous solution containing TBC constituents such as rare earths and zirconium in soluble forms (126). The aqueous solution is then filtered (128) to collect any remaining insoluble constituents, and the filtrate is collected for further processing of the aqueous reaction solution as will now be described.

### Separation and Recovery of Zirconium (Low pH) (136)

The collected filtrate reaction solution concentration is first adjusted to a concentration of approximately 2% to 20% rare earth+zirconium in solution, and more preferably 5% to 12% rare earth+zirconium in solution. By maintaining the reaction solution pH <3.0, methods including crystallization and precipitation may be utilized to produce solids consisting of high purity zirconium compounds (e.g., sulfate, fumarate, benzoate, phthalate, mandelate, and others), where the zirconium compounds would yield zirconia raw materials with the ZrO₂+HfO₂+REO purity of 99.0% to 99.99%. More specifically, the methods further disclosed herein enable the production of: zirconia products having a purity of greater than 99.9% (ZrO₂—HfO₂-REO) and a grain size suitable for sintering to ingot density, REO products having a purity of greater than 99.9% (REO-ZrO₂—HfO₂) and a grain size suitable for sintering to ingot density, and stabilized zirconia products having a purity of greater than 99.9% (ZrO₂—HfO₂-REO) and a grain size suitable for sintering to ingot density.

Next, the remaining reaction solution filtrate is filtered to separate the zirconium-containing solids, and the reaction solution filtrate is again collected for further processing of the reaction solution.

The solids collected from the filtering operation may be washed to further reduce impurities. Wash solutions can include solutions of ammonium sulfate, fumaric acid, benzoic acid, phthalic acid, mandelic acid, and others. The washed solids can be further processed to transform or convert the solids into alternate reactive zirconium chemicals, such as zirconium carbonate, hydrate, or nitrate. Washed solids are re-dispersed in water or reactive solutions at concentrations of 1% to 50%. Reactants used to convert the washed solids can include ammonium hydroxide, ammonium carbonate, and others. For example, ammonium hydroxide can be added to a dispersion of ammonium zirconium sulfate solids precipitated at low pH, 50% dispersion of solids in de-ionized water, to convert the solids into zirconium hydrate. While it is possible to use the crystallized/precipitated zirconium solids as reactive zirconium chemicals in these original forms, in most cases they would likely be converted into other reactive chemicals prior to use, for example, zirconium hydrates or carbonates. The washed solids can be reused in TBC production as reactive zirconium chemicals or converted to high purity monoclinic zirconia via calcination (174).

### Separation and Recovery of Zirconium (Moderate pH) (146)

Next, the pH of the remaining reaction solution is again adjusted, but to a moderate pH of 3-4 using ammonium hydroxide or another base, with the addition of the base causing zirconium hydrate to precipitate out of the remaining reaction solution. The pH adjusted reaction solution is then filtered to separate the zirconium hydrate precipitate from the remaining reaction solution filtrate, which is collected for further processing of reaction solution. The collected zirconium hydrate solids can be washed to further reduce impurities, and the wash solutions can include deionized water (174). The washed zirconium hydrate solids can be reused in TBC production as reactive zirconium chemicals, or they can be converted to high-purity monoclinic zirconia via calcination.

### Separation and Recovery of Rare Earths

To separate the rare earth, the pH of remaining reaction solution is now adjusted to approximately 6.0 using ammonium hydroxide or another base. This will induce the precipitation of any excess zirconium constituents and various impurities. This precipitate will represent a small fraction of the overall TBC waste materials, as most of the zirconium constituents were recovered at higher purities (ZrO₂+HfO₂+REO purity≥99.0% to 99.99%) in the lower pH separations described above. Once again, the reaction solution filtrate is filtered to separate the zirconium hydrate precipitate and associated impurities (156). At this stage, the collected zirconium hydrate precipitate will possess lower purity due to the presence of various other contaminants which also precipitate at the higher pH level, rendering this portion of the recovered waste unsuitable for use in TBC applications. However, the low purity zirconium may be collected for non-TBC applications (194).

Next the collected reaction solution filtrate is further processed by the addition of oxalic acid to the reaction solution to precipitate the rare earth constituents (e.g., rare earth oxalate) at high purities. At pH levels in the range of about 1 to about 3 pH, various rare earths are precipitated. These rare earth constituent precipitates are then filtered to separate the rare earth oxalate precipitate from remaining reaction solution (166). The collected rare earth oxalate precipitate is then washed to further reduce impurities. Suitable wash solutions can include dilute oxalic acid solution, and the wash solutions may be heated to enhance removal of impurities. The washed solids can be further processed to convert the solids into reactive rare earth chemicals, such as rare earth carbonate or nitrate. Reactants used to convert the washed solids can include ammonium carbonate and others. The washed rare earth oxalate solids can be converted to rare earth oxides via calcination and then reused in TBC production.

As one example, producing rare earth compounds from recycled TBC waste, may include rare earth oxalate produced by the following process. Initially, the aqueous reaction solution is produced, with the solution consisting of water-soluble rare earth and zirconium constituents dissolved in water at concentrations ranging from 2% to 20% and a pH below 3. The reaction solution is then treated to elicit the formation of zirconium compound solids from the solution at a pH below 3 using separation methods consisting of: ammonium zirconium sulfate precipitation, zirconium-organic acid compound precipitation or others. The precipitated zirconium compound solids are then separated from the remaining solution using one or more methods that may include filtration. The solids may be further processed for reuse according to the other disclosure herein. Next, the remaining reaction solution pH is adjusted to above 3, and more preferably 5-6, using a basic reactant, such as ammonium hydroxide, to elicit the formation of solid compounds comprising the residual zirconium and trace contaminant constituents, such as zirconium and trace contaminant hydrate compounds. The precipitated residual zirconium and trace contaminant compound solids are separated from the remaining solutions, once again by any number of methods which may include filtration. These materials can be discarded or used in lower purity/non-TBC applications. The remaining reaction solution pH is adjusted to above 6, more preferably from 6 to less than 9 using a basic reactant, such as ammonium hydroxide. The rare earth precipitates at a pH of 9, so it is preferably to stay just below this level. The remaining reaction solution is treated with a rare earth precipitating agent to elicit the formation of a rare earth compound via precipitation, with the rare earth precipitating agent consisting of oxalate-containing compounds such as oxalic acid or ammonium oxalate, and the precipitated rare earth compound solids are then separated from the remaining reaction solution, again by filtration or equivalent methods. Once separated, the precipitated rare earth compound solids are washed using wash solutions selected from the group of oxalic acid and ammonium oxalate (174).

As noted previously, the precipitates formed during the processes disclosed above may also be converted to carbonates, hydrates or nitrates (176). And similar operations may be applied to the rare earth precipitates. As one example, ammonium zirconium sulfate may be produced from recycled TBC waste by the following operations. First, the aqueous reaction solution is produced, with the solution consisting of water-soluble rare earth and zirconium constituents dissolved in water at concentrations ranging from 2% to 20% and a pH below 3, and then the solution concentration and pH conditions are maintained for a period of time (e.g., from 1 to 24 hours), during which ammonium zirconium sulfate will precipitate from the concentrated solution. The precipitated or crystallized solids are then separated from the remaining solution by methods such as filtration, and the separated solids are washed using wash solutions including an ammonium sulfate solution.

As another example, zirconium-organic acid compounds including fumarate, benzoate, phthalate, lactate, and mandelate may be produced using recycled TBC waste, by the following operations. Initially an aqueous reaction solution is produced, with the solution consisting of water-soluble rare earth and zirconium constituents dissolved in water at concentrations ranging from 2% to 20% and a pH below 3. The solution pH is then adjusted to between 2-3 using basic reactant, which may include ammonium hydroxide. Next the reaction solution is treated with an organic acid to elicit the formation of a zirconium-organic acid compound or precipitate via precipitation, with an organic acid selected from the group of acids consisting of fumaric acid, benzoic acid, phthalic acid, lactic acid and mandelic acid. The precipitated zirconium-organic acid compound solids are separated from the remaining solution by methods such as, but not limited to, filtration, and the solids are then washed using wash solutions selected from a group of acids consisting of fumaric acid, benzoic acid, phthalic acid, lactic acid and mandelic acid.

Alternate reactive zirconium chemicals may similarly be produced from recycled TBC waste, including zirconium carbonate, hydrate, and nitrate by the following operations. The zirconium-containing solids are washed and dispersed in de-ionized water. Next, the dispersed zirconium-containing solids are treated to convert the solids in accordance with a reaction selected from the group consisting of: (i) reacting the solids with ammonium carbonate to produce zirconium carbonate as converted zirconium solids, (ii) reacting the solids with ammonium hydroxide to produce zirconium hydrate as converted zirconium solids, or (iii) reacting the solids with nitric acid to produce zirconium nitrate as converted zirconium solids. The converted zirconium solids are then separated from the remaining solution, for example by filtration, and the separated solids are washed using a wash solution, which may be selected from the group consisting of ammonium carbonate and ammonium hydroxide.

### Production of Thermal Barrier Coating Materials from Recycled TBC Wastes—EB-PVD Ingots

Recycled TBC EB-PVD ingots can be produced from zirconium chemicals, monoclinic zirconia, and rare earth oxide recycling products. For example, EB-PVD ingot materials can be prepared from a mixture of monoclinic zirconia and rare earth oxide powders; or, more preferably, the EB-PVD ingot materials can be prepared from a mixture of monoclinic zirconia and rare earth oxide-stabilized zirconia powders, where the rare earth oxide-stabilized zirconia powder is produced using rare earth and zirconium chemical raw materials.

The rare earth oxide-stabilized zirconia can be produced by the reaction of a recycled zirconium chemical raw material (e.g., ammonium zirconium sulfate, zirconium fumarate, zirconium carbonate, zirconium hydrate, zirconium nitrate, zirconium benzoate, zirconium phthalate, zirconium lactate, zirconium mandelate, and others) with a recycled rare earth raw material (rare earth oxide, rare earth carbonate, rare earth nitrate, and others), resulting in the product consisting of a chemical mixture of rare earth and zirconium which can be converted to a rare earth oxide-stabilized zirconia via calcining. Prior to calcining, the stabilized zirconium and monoclinic zirconia raw materials should be screened with a sieve, preferably 10 mesh or below, to provide particle uniformity and adequate compaction density for the ingots. Grain size or, more accurately, particle size of the raw material components required for use in ingots is not necessarily fixed. Ingots possessing the proper density can be produced from powders of varying particle sizes by modifying the sintering temperature (and, to a lesser degree, the pressure at which the ingot is formed). Correspondingly, the raw material particle size distribution/surface area will determine the sintering temperatures required to reach the desired density (typ. (1000-1700° C.).

Subsequently, calcining the stabilized zirconium raw material (184), at temperatures between 800° C. and 1700° C., converts to rare earth oxide-stabilized zirconia and modifies the physical powder properties (e.g., particle size distribution, specific surface area) to preferential levels. And, calcining the monoclinic zirconia raw material, at temperatures between 800° C. and 1700° C., modifies the physical powder properties (e.g., particle size distribution, specific surface area) to preferential levels. Post-calcining, the process then screens the rare earth oxide stabilized zirconia and monoclinic zirconia powders to at least below 10 mesh particle size.

Next, the screened stabilized zirconia and monoclinic zirconia powders are blended to achieve a desired EB-PVD ingot material composition. The blended powder may then be formed into an EB-PVD ingot body by compacting the blended powder (190). In one preferred embodiment the blended powder is formed into an ingot via cold isostatic pressing. The cold-pressed ingot is then heated to temperatures of 1000° C. and 1700° C. to yield a sintered ingot, having approximately 50% to 70% of theoretical density for the given composition, with open porosity. For example, yttria-stabilized zirconia ingots should be heated to temperatures which result in the sintered ingot possessing a density exceeding 3.6 g/cm³. The sintered ingot may then be machined to final desired dimensions for the EB-PVD ingot.

### Production of Thermal Barrier Coating Materials from Recycled TBC Wastes—Plasma Spray

Recycled TBC plasma spray materials can be produced from zirconium chemicals, monoclinic zirconia, and rare earth oxide recycling products. In one embodiment, the plasma spray materials can be prepared (192) from a mixture of monoclinic zirconia and rare earth oxide powders, or more preferably, the plasma spray materials can be prepared from rare earth oxide-stabilized zirconia raw materials. And, the rare earth oxide-stabilized zirconia raw materials can be formed by either thermal (fusion) or chemical methods.

The thermal preparation of the rare earth oxide-stabilized zirconia can be achieved by heating a mixture of recycled rare earth oxide and monoclinic zirconia raw materials to greater than the melting point of each component (at least approximately 2800° C.), allowing the materials to melt and react into a stabilized mixture; thermal preparation methods include induction and electric arc fusion.

The chemical preparation of rare earth oxide-stabilized zirconia can be achieved by the reaction of a recycled zirconium chemical raw material (ammonium zirconium sulfate, zirconium fumarate, zirconium carbonate, zirconium hydrate, zirconium nitrate, zirconium benzoate, zirconium phthalate, zirconium lactate, zirconium mandelate, and others) with a recycled rare earth raw material (rare earth oxide, rare earth carbonate, rare earth nitrate, and others), resulting in the product consisting of a chemical mixture of rare earth and zirconium which can be converted to a rare earth oxide-stabilized zirconia via calcining. And, stabilized zirconia plasma spray materials can be further processed to achieve desired TBC material properties, including milling, screening, spray drying, and firing to produce final TBC products.

It should be understood that various changes and modifications to the embodiments described herein will be apparent to those skilled in the art. Such changes and modifications can be made without departing from the spirit and scope of the present disclosure and without diminishing its intended advantages. It is therefore anticipated that all such changes and modifications be covered by the instant application.

What is claimed is:

1. A method for recovery of thermal barrier coating materials from waste comprising:
collecting and classifying TBC waste materials to determine chemical composition and impurity levels;
processing the TBC waste to convert it to a fine powder;
reacting the fine powder to form a soluble zirconium- and rare earth-containing material; and
collecting the soluble zirconium and rare earth containing materials, including recycled zirconium and rare earth materials, which consist essentially of precursor materials suitable for use in producing TBC feedstock materials.

2. The method according to claim 1, wherein the resulting precursor materials are calcined into oxides, pressed into raw EB-PVD ingots, fired, and machined to form finished EB-PVD ingots.

3. The method according to claim 1, wherein the resulting precursor materials are calcined into oxides and further processed into plasma spray powder materials.

4. The method according to claim 1, wherein processing the TBC waste to convert it to a fine power includes physically or mechanically altering the waste material to produce a powder having particle sizes equal to or less than 841 to 44 microns.

5. The method according to claim 1, wherein reacting the fine powder includes reacting the fine powder by thermal or chemical methods.

6. The method according to claim 1, wherein the fine powder consists essentially of water-soluble zirconium and rare earth-containing material, and where reacting the fine powder includes reacting the fine powder using a sulfate reaction process.

7. The method according to claim 6, further comprising the following steps:
dissolve the water-soluble zirconium- and rare earth-containing powder in water to form a reaction solution containing both zirconium and rare earth constituents;
remove any insoluble materials present within the reaction solution;
adjust the reaction solution pH and chemistry to extract zirconium compounds at low pH;
collect and process zirconium compounds from the reaction solution;
further adjust the remaining reaction solution pH and chemistry to extract residual contaminants;
remove residual contaminants from the remaining reaction solution;
further adjust the remaining reaction solution pH and chemistry to extract rare earth compounds;
collect and process the rare earth compounds to yield materials suitable for use in TBC materials.

8. The method according to claim 1, further including producing raw materials for EB-PVD or plasma spray applications utilizing recycled TBC waste, comprising an operation selected from the group consisting of:
a. a portion of the raw materials are produced using ammonium zirconium sulfate recycled from TBC wastes;
b. a portion of the raw materials are produced using zirconium-organic acid compounds recycled from TBC wastes, including at least one of zirconium fumarate, benzoate, phthalate, lactate, and mandelate;
c. a portion of the raw materials are produced using crystallized or precipitated zirconium compounds from (a) and (b), which are converted into other reactive zirconium chemicals, including at least one of zirconium carbonate, hydrate, and nitrate;
d. a portion of the raw materials are produced using rare earth oxalate compounds recycled from TBC wastes;
e. a portion of the raw materials are produced using precipitated zirconium compounds from (d) which are converted into other reactive rare earth chemicals, including at least one of rare earth carbonate and nitrate; and
f. a portion of the raw materials are produced using crystallized, precipitated, or converted zirconium compounds from (a), (b), (c), (d) and (e) which are calcined to oxides, including at least one of zirconia and rare earth oxides.

9. The method according to claim 7, further including producing ammonium zirconium sulfate from recycled TBC waste by the following operations:
a. the aqueous reaction solution is produced, with the solution consisting of water-soluble rare earth and zirconium constituents dissolved in water at concentrations ranging from 2% to 20% and a pH below 3;
b. maintaining the solution concentration and pH conditions for a period of 1 to 24 hours, during which ammonium zirconium sulfate will precipitate from the concentrated solution;
c. separating the precipitated/crystallized solids from the remaining solution; and
d. washing the separated solids using wash solutions including ammonium sulfate solution.

10. The method according to claim 7, further including producing zirconium-organic acid compounds from recycled TBC waste, including fumarate, benzoate, phthalate, lactate, and mandelate by the following operations:
a. the aqueous reaction solution is produced, with the solution consisting of water-soluble rare earth and zirconium constituents dissolved in water at concentrations ranging from 2% to 20% and a pH below 3;
b. adjusting the solution pH to between 2-3 using basic reactant;
c. treating the reaction solution with an organic acid to elicit the formation of a zirconium-organic acid compound via precipitation, with the organic acid selected from the group of acids consisting of fumaric, benzoic, phthalic, lactic, and mandelic;
d. separating the precipitated zirconium-organic acid compound solids from the remaining solution; and
e. washing the solids using a wash solution selected from the group of acids consisting of fumaric, benzoic, phthalic, lactic, and mandelic.

11. The method according to claim 9, further including producing alternate reactive zirconium chemicals from recycled TBC waste, including zirconium carbonate, hydrate, or nitrate by the following operations:
a. the washed zirconium-containing solids are dispersed in de-ionized water;
b. treating the dispersed zirconium-containing solids to convert the solids in accordance with a reaction selected from the group consisting of: reacting the solids with ammonium carbonate to produce zirconium carbonate as converted zirconium solids, reacting the solids with ammonium hydroxide to produce zirconium hydrate as converted zirconium solids, or reacting the solids with nitric acid to produce zirconium nitrate as converted zirconium solids;
c. separating the converted zirconium solids from the remaining solution; and
d. washing the separated solids using wash solutions selected from the group of wash solutions consisting of ammonium carbonate, and ammonium hydroxide.

12. The method according to claim 10, further including producing alternate reactive zirconium chemicals from recycled TBC waste, including zirconium carbonate, hydrate, or nitrate by the following operations:
a. the washed zirconium-containing solids are dispersed in de-ionized water;
b. treating the dispersed zirconium-containing solids to convert the solids in accordance with a reaction selected from the group consisting of: reacting the solids with ammonium carbonate to produce zirconium carbonate as converted zirconium solids, reacting the solids with ammonium hydroxide to produce zirconium hydrate as converted zirconium solids, or reacting the solids with nitric acid to produce zirconium nitrate as converted zirconium solids;
c. separating the converted zirconium solids from the remaining solution; and
d. washing the separated solids using wash solutions selected from the group of was solutions consisting of ammonium carbonate, and ammonium hydroxide.

13. The method according to claim 7, further including producing rare earth compounds from recycled TBC waste, including rare earth oxalate by the following operations:
a. producing an aqueous reaction solution with the solution consisting of water soluble rare earth and zirconium constituents dissolved in water at concentrations ranging from 2% to 20% and a pH below 3;
b. treating the reaction solution to elicit the formation of zirconium compound solids from the solution at a pH below 3 using separation methods consisting of: ammonium zirconium sulfate precipitation, zirconium-organic acid compound precipitation, and others
c. separating the precipitated zirconium compound solids from the remaining solution;
d. adjusting the pH of the remaining reaction solution to above 3 using a basic reactant to elicit the formation of solid compounds consisting of the residual zirconium and trace contaminant constituents;
e. separating the precipitated residual zirconium and trace contaminant compound solids from the remaining solutions;
f. adjusting the pH of the remaining reaction solution to above 6 using a basic reactant
g. treating the pH adjusted remaining reaction solution with a rare earth precipitating agent to elicit the formation of a rare earth compound via precipitation, where the rare earth precipitating agent consists essentially of oxalate-containing compounds;
h. separating the precipitated rare earth compound solids from the remaining reaction solution; and
i. washing the solids using wash a solution.

14. The method according to claim 1, further including calcining zirconium compounds to oxides, including at least one of a zirconium oxide and a rare earth oxide.

15. The method according to claim 1, further including producing zirconia products having a purity of greater than 99.9% (ZrO₂—HfO₂-REO) and a grain size suitable for sintering to ingot density.

16. The method according to claim 1, further including producing REO products having a purity of greater than 99.9% (REO-ZrO₂—HfO₂) and a grain size suitable for sintering to ingot density.

17. The method according to claim 1, further including producing stabilized zirconia products having a purity of greater than 99.9% (ZrO₂—HfO₂-REO) and a grain size suitable for sintering to ingot density.
